(19) United States
(12) Patent Application Publication
(10) Pub. No.: US 2022/0202276 A1
(43) Pub. Date: Jun. 30, 2022

(54) ENDOSCOPE VERTEBRAE

(71) Applicant: Boston Innovation Technology Corporation, Waltham, MA (US)

(72) Inventors: Dwight Alan MEGLAN, Westwood, MA (US); Yunfei Richard CAO, Lincoln, MA (US)

(73) Assignee: Boston Innovation Technology Corporation, Waltham, MA (US)

(21) Appl. No.: 17/134,422

(22) Filed: Dec. 27, 2020

(51) Int. Cl. A61B 1/005; A61B 1/018; A61B 1/00; A61B 1/05; A61B 1/06

(52) U.S. Cl. CPC A61B 1/0056; A61B 1/0052; A61B 1/0057; A61B 1/018; A61B 1/00117; A61B 1/05; A61B 1/06; A61B 1/00114; A61B 1/0011

(57) ABSTRACT

Herein disclosed is an endoscope that includes a handle and an elongated probe having a distal end and a proximal end. The handle abuts the proximal end of the probe and includes an articulation lever. The probe also includes a vertebrae column immediately abutting the distal end, on which at least one sensor is amounted. The vertebrae column is configured to have at least two parallel groups of gaping slits, along an axial direction of the vertebrae column. Each two of the axially adjacent gaping slits come from the two groups of gaping slits respectively and are juxtaposed in circumferential positions in the respective circumferential planes. The articulation lever and the distal end are connected by pulling wires, and when the articulation lever is maneuvered, the distal end is pulled away from the axial direction, causing the vertebrae column to deflect.

### TECHNICAL FIELD

This application relates generally to endoscopes, particularly to endoscopes with vertebrae distal portion that deflects upon being pulled, and further to methods of the same for configuring and using the endoscopes.

### BACKGROUND

Generally, the cost of medical procedures for minimally, invasively exploring and treating the body of a subject is significantly impacted by the cost of the endoscopes or scope used. Such scopes can be used, for example, for imaging and treating issues involving the kidney calyces, bladder, and ureter. In addition to the significant cost of such reusable scopes, which are on the order of about $25,000 each piece, they must be cleaned and sterilized after each procedure to prevent cross infection of patients. This process has been shown to take on the order of 4 hours with the cost of material or servicing the material being over $1000. For a high throughput endoscopy center, this means having to stock several scopes to do multiple procedures in a day as well as having to stock additional scopes to supplement the instrument due to breakage. It has been documented that these types of scopes need repair after 8-12 uses. In addition, for resterilization, there is clear evidence in recent years that many scopes are not as sterile as required after processing, such that cross infection still occurs. Resterilization in any form is a challenge in austere and cost sensitive environments, such as in the developing world.

The distal portion of endoscopes are required to be stiff enough to be pushed through cavities or conduits of organs, yet passively flexible to deflect when manipulated by the control attached to the handle. In existing practice, the distal portion of endoscopes are made of medical grade and expensive metal, such as steel, tungsten, platinum, or the like, in a shape of an elongated conduit. Metal mesh or metal tube with complicated perforation maybe applied. Further, in existing practice, internal working channels need to be built or placed into the metal conduit as an extra step. The cost of material and manufacturing process is high. In an economic sense, the existing type of distal portion of endoscopes does not allow it to be disposed after only one-time usage, and therefore does not allow the application of disposable or partially disposable endoscopes.

A traditional endoscope which is used for many times being sterilized between uses also has other significant disadvantages, including high sterilization cost and high risk of insufficient sterilization. In addition, clinicians have specific expectations as to how an ureteroscope should behave mechanically when they manipulate it. As a result, meeting the high quality of clinician expectations and while also achieving low cost to make single use economically possible are often the two conflicting factors not successfully reconciled in the existing practice. Therefore, producing high quality single use ureteroscope whose economics are compatible with typical hospital and outpatient clinics is highly desirable.

For these reasons, being able to eliminate the need for resterilization, while also reducing the cost of endoscopy use per procedure and meeting clinician quality expectations, can lead to improvements in both patient safety and hospital efficiency. Accordingly, herein disclosed methods and apparatus are directed to solve one or more problems set forth above and other problems.

### SUMMARY

In accordance with a first aspect of the present disclosure, there is set forth an endoscope that includes an elongated probe having a sensor at a distal end of the probe, and a vertebrae column immediately abutting the distal end. The endoscope further includes a handle abutting a proximal end of the probe and comprising an articulation lever.

The vertebrae column is configured to have at least two parallel groups of gaping slits, along an axial direction of the vertebrae column. Each two axially adjacent gaping slits come from the two groups of gaping slits respectively, are juxtaposed in circumferential positions in the respective circumferential planes. Furthermore, the articulation lever and the distal end are connected by pulling wires, and when the articulation lever is maneuvered, the distal end is pulled away from the axial direction, causing the vertebrae column to deflect.

The vertebrae column also envelopes working channel, pull wire channel and likely optical fiber cable. The vertebra column can be made of plastic molding or protrusion molding, with the working channel, pull wire channel and the optical fiber cable all formed or embedded at the same time when the vertebra column is formed during the plastic molding process. The vertebra gaping slits can be machined onto the vertebra column or directed molded together with the vertebra column.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the disclosed systems and methods and are not intended as limiting. For purposes of clarity, not every component may be labeled in every drawing. In the following description, various embodiments are described with reference to the following drawings.

FIG. 1 is a perspective view of an endoscope with a vertebra column at the distal portion in accordance with the present disclosure.

FIG. 2 is a side view of the elongated vertebra column of the endoscope in accordance with the present disclosure.

FIG. 3A and FIG. 3B are circumferential cross-section views of the vertebra column of the endoscope in accordance with the present disclosure.

FIGS. 4-7 are circumferential cross-sectional views of the vertebra column of the endoscope in accordance with the present disclosure.

FIG. 8 is longitudinal cross-sectional views of the vertebra column of the endoscope in accordance with the present disclosure.

FIG. 9 is a side view of the elongated vertebra column of the endoscope in accordance with the present disclosure.

FIG. 10 and FIG. 11 are two detailed longitudinal cross-sectional views of the vertebra column of the endoscope in accordance with the present disclosure.

FIG. 12 is longitudinal cross-sectional view of part of the handle of the endoscope and the elongated probe in accordance with the present disclosure.

FIG. 13A is a side view of the elongated vertebra column of the endoscope with a bent distal tip in accordance with the present disclosure.

FIG. 13B is an elaborated view of the vertebrae column 12 surrounding one vertebrae slit 10 in accordance with the present disclosure.

FIG. 14 is a diagram showing the sizing of the vertebrae slit and the bending of the distal tip of the endoscope in accordance with the present disclosure.

### DETAILED DESCRIPTION

### List of Nomenclatures

Numerals corresponding to those shown in the drawings are listed in Table-1. Terms assigned to corresponding numerals are also given in Table-1.

TABLE 1
Numerals and corresponding terms used in the present disclosure.

| | | | |
|---|---|---|---|
| 10a | first vertebrae slit | 20 | working channel |
| 10b | second vertebrae slit | 22 | coil spring |
| 10 | vertebrae slit | 24 | gap |
| 12 | vertebra column | 26 | working pulley |
| 120 | vertebra sheathing | 28 | optical fiber cable |
| 12b | probe conduit | 30 | camera |
| 14 | pull wire | 32 | electrical wire |
| 16 | distal tip | 34 | bridge |
| 18 | pull wire channel | 36 | pointing guide |
| 38 | pull wire holder | 44 | articulation lever |
| 42 | endoscope handle | 48 | handle |
| 46 | probe | 52a, b | working channel adaptor |
| 50 | articulation lever | 56 | probe proximal end |
| 54 | working pully | 80 | arch |
| 60 | handle coupling head | 100 | endoscope |

The following description of the vertebra column of the endoscopes uses an ureteroscope as an example. It should be appreciated that the scope and spirit of this disclosure is not limited to this example. The example of using partially reusable endoscopes, entirely reusable, or entirely disposable endoscopes do not affect the scope of present disclosure. The term of endoscope or scope can be interchangeable used with many types of endoscopes, such as ureteroscope, cystoscope, bronchoscope, and laparoscope, etc.

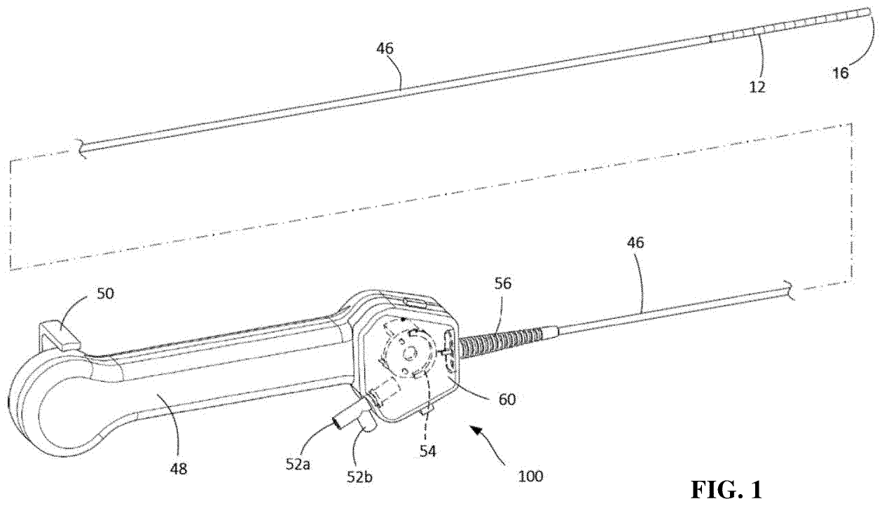

FIG. 1 is a perspective view of the endoscope 100 in accordance of the present disclosure. Endoscope 100 includes a probe handle 48 and a probe 46 which is exchangeably called an elongated probe 46 as it often takes an elongated shape to assist the insertion into human or animal bodies to provide investigation or treatment for the targeted organs.

Referring to FIG. 1, probe 46 may be an elongated conduit or tube, often being flexible. Probe 46 may include a probe dismal portion 12 called vertebra column 12 and a distal tip 16 (both described later in greater detail). Vertebra column 12 is often rigid enough to be pushed through to guide the probe to enter into body cavities or organs, yet passively flexible to change direction to where the inspection is needed to take place. Probe 46 may also have a proximal portion 56 connecting to handle 48. Endoscope 100 further includes an articulation rudder 50 positioned at handle 48, which upon being maneuvered, causes a deflection of vertebra column 12, which is connected with the articulation rudder 50 by a pulling assembly explained later in details.

Reference is still made to FIG. 1. In some embodiment, endoscope 100 includes a handle coupling section 60 configured to facilitate the attachment and detachment of handle 48 and probe 46 upon correspondingly at starting and completion of each clinical use, resulting in the convenient attachment and detachment of the optical, electrical, and mechanical functions without causing contamination to the reusable handle 48. The details of this embodiment are described in a co-pending U.S. patent application Ser. No. 16/865,593, filed on May 4, 2020, the entire content of which is herein incorporated by reference.

In various embodiments, part of the endoscope is disposable. For example, the coupling section 60 can be part of the handle and/or any part of the probe. All such variations are within the scope of the present disclosure. handle 48 is reusable, while probe 46 is detachable from the handle so that it may be of single use.

Optical, mechanical, and electrical transmissions are provided from distal tip 16 to probe handle 48. Detailed description regarding the transmission of optical, electrical, and mechanical functions between handle 48 and probe 46 is provided hereinafter.

FIG. 2 is a side view of the elongated probe of the endoscope in accordance with of the present disclosure.

Referring to FIG. 2, probe 46 includes distal tip 16 and vertebrae column 12. Probe 46 has an axial direction along the probe's elongated body, and a circumferential direction which is any plane perpendicular to the axial direction. Vertebrae column 12 includes a plurality of vertebrae slits 10. In one example embodiment, slits 10 are separated into two groups, a first group of vertebrae slits 10A and a second group of vertebrae slits 10B, each group is arranged to be along a longitudinally line on the surface of vertebrae column, largely parallel of each other, and parallel of the axial direction of probe 46.

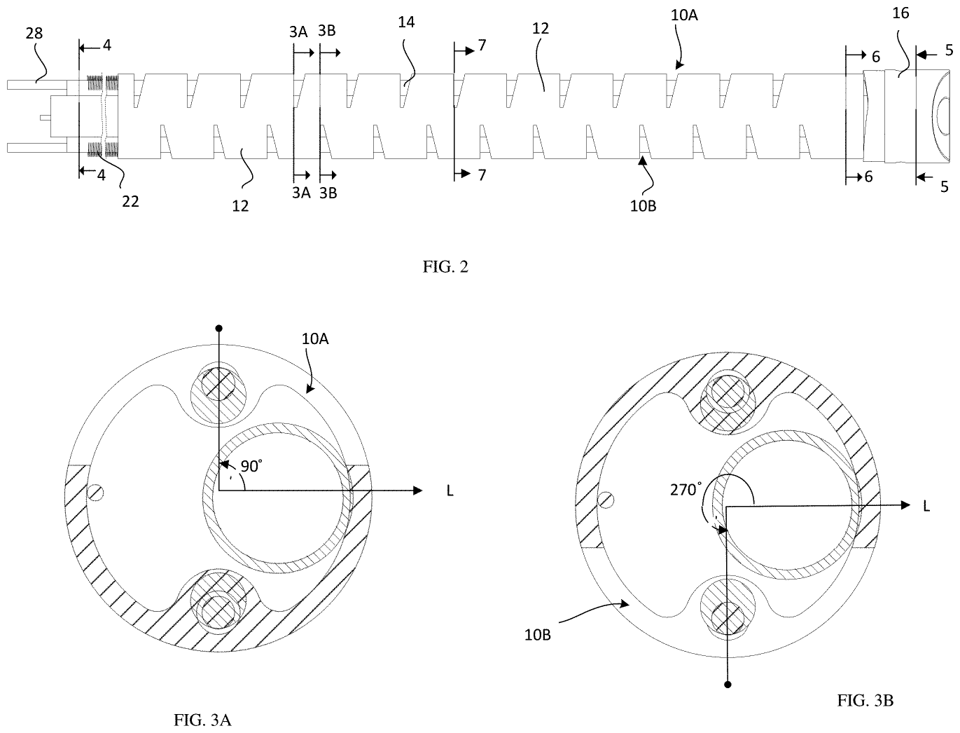

Each two axially adjacent gaping slits come from the two groups of gaping slits respectively, are juxtaposed in circumferential positions in the respective circumferential planes. That is to say, in any two adjacent circumferential cross-sections cut at the two respective vertebra slits, such as along cut lines 3A and 3B, the two vertebra slits 10A and 10B can be juxtaposed as shown in FIGS. 3A and 3B.

Referring to FIGS. 3A and 3B, two polar coordinates are shown. The circumferential center points of the planes cut at lines 3A and 3B are the respective origins of the polar coordinates. The pole, or the ray from the origin in the reference direction is the polar axis. The distance from the pole is called the radial coordinate, radial distance or simply radius, and the angle is called the angular coordinate, polar angle, or azimuth. The polar coordinate is often denoted by the radius r or ρ, and the angular coordinate by φ, θ, or t. Angles in polar notation are generally expressed in either degrees or radians (2π rad being equal to 360°). In FIGS. 3A and 3B, for example, the two polar coordinates are axially apart with an off distance between circumferential planes cut lines 3A and 3B respectively. In the polar coordinates, the center of slit 10A is at 90°, and the center of slit 10B is at 270° in the respective polar coordinates, with a 180° degrees between the two adjacent slits. It should be appreciated that the angular difference can be in any other degrees, to make the slits 10A and 10B in circumferentially juxtaposed positions, for example 120°. All such variations are within the scope of the present disclosure.

It can be appreciated that the juxtaposed positions in circumferential planes and interposed in axial direction of slit groups 10A and 10B provide the flexibilities at multiple circumferential directions, without harming the integrity of the vertebra column 12.

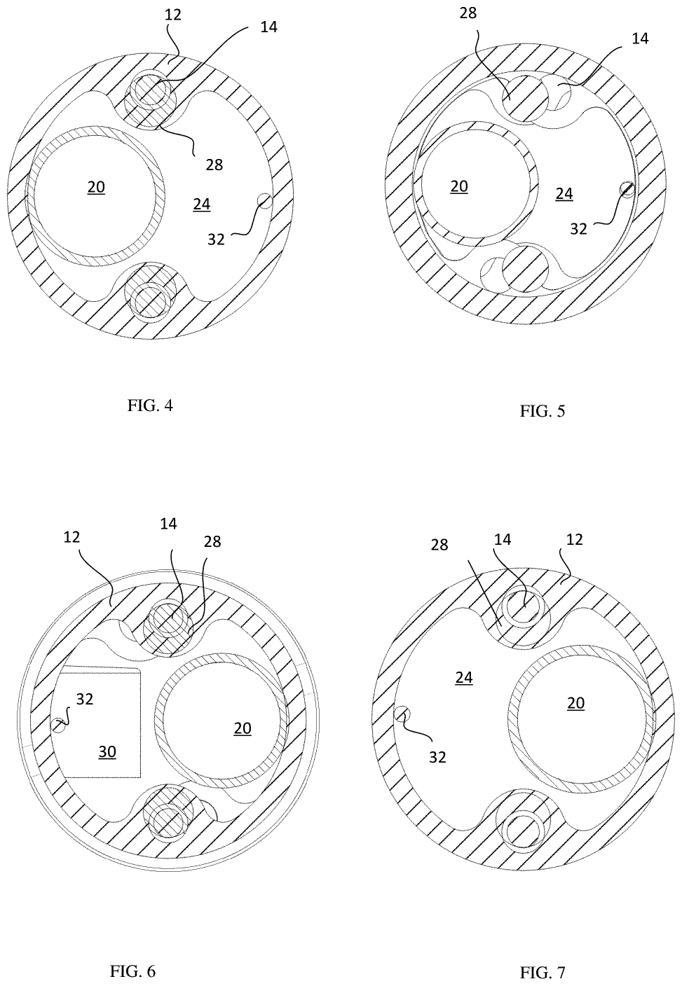

Reference now is made to FIGS. 4-7, which are cross-section views of the vertebra column 12. Continuing to refer to FIG. 2, FIG. 4 is a cross-section view viewed at a cross section at line 4-4 in FIG. 2, with viewing direction indicated in FIG. 2. FIG. 5 is a cross-section viewed at a cross section at line 5-5 in FIG. 2, with viewing direction indicated. FIG. 6 is a cross-section viewed at a cross section at line 6-6 in FIG. 2, with viewing direction indicated. FIG. 7 is a cross-section viewed at a cross section at line 7-7 in FIG. 2, with viewing direction indicated in FIG. 2. As can be seen, the relative positions of different elements included in vertebra column 12 is shown at different circumferential planes.

As shown in FIGS. 6 and 7, a working channel 20 is positioned within space gap 24, which can be premade together with the vertebra column or later inserted directly into space gap 24. Electric wire 32 is also deposited at the inner surface of vertebra column 12 and in space gap 24. As can been seen, optical fiber cable channel 28 and a pull wire channel 18 and space gap 24 can all be premade or made together with vertebra column 12 in a process of plastic molding or plastic extrusion. This is different from existing practice, in which when metal material is used, at least the working channel and pull wire channel are not practical to be manufactured together in one process with the vertebra column.

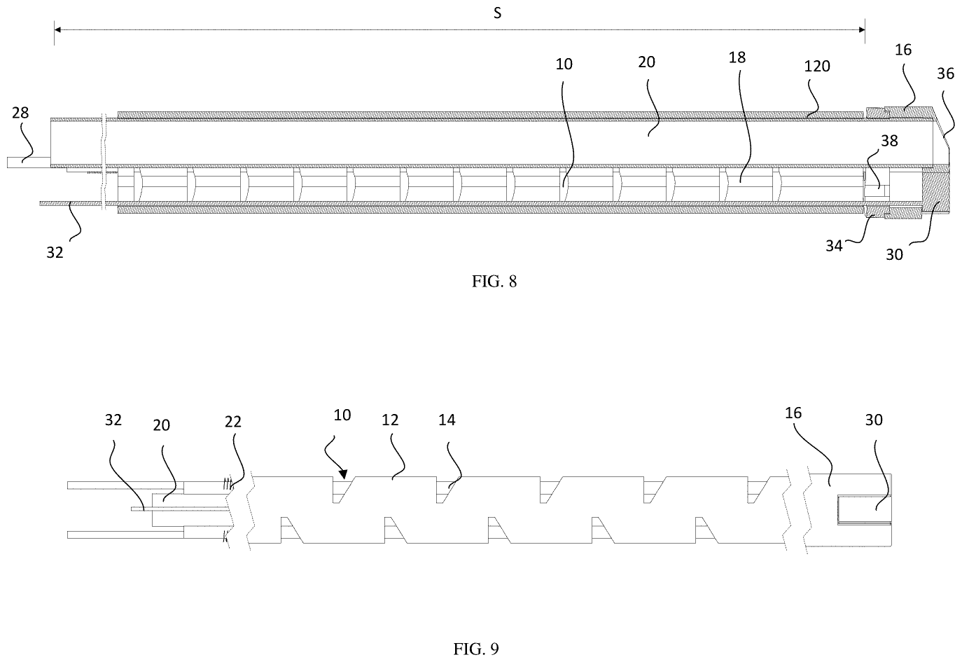

FIG. 8 is a longitudinal cross-section view of the probe of the endoscope 100 in accordance with of the present disclosure. As shown in FIG. 8, probe 46, as well as vertebra column 12 as a continuous part of probe 46, comprises pull wire channel 18, a working channel 20, an electrical wire 32 and optical fiber cable 28. In some embodiment, probe 46 also includes vertebra sheathing 120. Distal tip 16 includes a slanted pointing guide 36 at its front face, serving to ease and guide the inserting of probe 46.

Distal tip 16 includes a pull wire holder 38 configured to hold pull wire 14 threaded through pull wire channel 18. Pull wire holder 38 may be as simple as a holed short cylinder, allowing pull wire 14 to be threaded through, the size of which is configured to be smaller than a knot (not shown) tied by the end of pull wire 14. Alternatively, pull wire holder 38 may be a spring-loaded clamp that can be opened and closed to hold the end of pull wire 14. Further alternatively, pull wire 14 with one end having a crimp of a diameter larger than the hole of wire holder 38 can be threaded into the wire holder 38 from the other smaller end. All such alternations are within the scope of the present disclosure.

Further at distal tip 16, probe 46 also includes a bridge 34 connecting distal tip 16 with the main body of vertebra column 12.

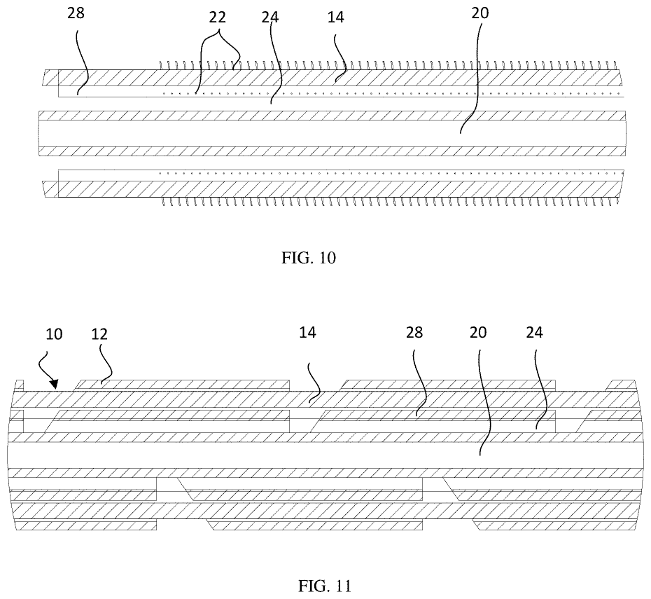

Referring to FIGS. 2, 9, 10 and 11, vertebrae column 12 also includes a pair of coil springs 22 configured to cause tension in the axial direction of the body of probe 46 so that pull wire 14 does not drift idle across the cross section of the main body of probe 46. Preferably, each of the coil springs 22 is attached to the either end (the distal end or proximal end) of the main body of probe 46, encircling the corresponding pull wire 14.

As can been seen in FIGS. 2, 9, 10 and 11, vertebrae column 12, in axial direction, includes working channel 20 for hosting external tools, tubes and hoses to be delivered to or from distal tip 16. Vertebrae 12, in axial direction, envelops gap space 24, channels for optical fiber cable 28, electrical wire 32, and pull wire channel 18. The details on the electric wire 31 and fiber cable 28 is explained in the description that follows.

Referring to FIGS. 1 and 12, the mechanical transmission of movement from articulation rudder 50, being operated by an operator, to distal tip 16 is shown. Handle 48 or an interface part of the handle connecting with probe 46 includes a working pulley 54. Pull wire 14, may be looped around working pulley 54 or belts around half circumference of working pulley 54 on the side opposite to the entrance of probe proximal end 56, before entering into probe proximal end 56, then continues into pull wire channel 18 inside vertebra column 12. Pull wire 14 continues within pull wire channel 18 and reaches to distal tip 16 and attaches to pull wire holder 38 (see in FIG. 8) at distal tip 16. As such, when articulation rudder 50 is rotated, causing working pulley to swing, with pull wire being flexible but not stretchable, it causes pull wire 14 to pull distal tip 16, further causing a deflection of vertebra column 12.

Pull wire 14 may also be a pair of pull wires 14 correspondingly attached to either side of the working pulley 54 and two opposite positions of wire holder 38 at the distal tip 16. More often, there may be two wire holders 38 positioned at circumferentially opposite sides, or circumferentially juxtaposed positions. In another word, working pulley 54 and the distal end 16 can be cabled by a pair of working pull wires 14 in such a way to translate the motion or displacement of working pulley 54, causing distal end of the probe to be pulled off center. Subsequently, vertebrae column 12 is deflected to either one of the directions. The structure of pull wire 14 and working pulley 54 is in the fashion of belt pulley but with only one pulley, which is the working pulley 54. The other end of working pull wire is fixed inside the distal tip 16 on wire holder 38.

Therefore, once assembled, articulation rudder 50 causes motion or displacement via working pulley 54, through pull wire 14, further causing vertebra column 12 to deflect accordingly.

The pull wires 14 are constructed in a manner that enables them to be sufficiently flexible, so they do not interfere with the flex of the passively flexible portion. The pull wires must also be sufficiently strong to apply enough force to the steerable distal portion 16 such that it can be deflected. In some embodiment, these pull wires may be made of braided stainless steel in a 7×7×7 pattern and are placed symmetrically along a vertical axis of the probe 12 and working channel 20. As explained above, the pull wire's movement is controlled by a rotational articulation rudder 50 on the most proximal portion of the handle 48. Accordingly, if both sides were pulled equally, the tip will not deflect and instead will become more rigid from co-contraction.

Probe 12, in many embodiments, are rigidly flexible and distal tip 16 is steerable. The two threads of the loop of working pull wire 14 loop between working pulley 54 and the end of distal tip 16, so that a pull of pull wire 14 in any direction caused by the rotation of working pulley 54, results in a deflection of the tip (up to 270° in either direction from pointing forward). Pull wire 14 is arranged in a pull-pull setup in the steerable distal portion 16 such that the tip deflects toward the side which is being pulled.

As shown in FIGS. 6 and 9, a camera 30 is deposited at distal tip 16 at the end of the vertebra column 12 and connected with an electrical wire or electrical wires 32 which is threaded out to the probe conduit. Similarly, as shown in FIGS. 4-7, electric wire 32 is deposited at the inner surface of vertebra column 12 and in space gap 24 and threaded through the longitudinal probe 46.

Electric wire 32 may transmit data, such as image data via electric wire 32 to a processor (not shown) in handle 48. Alternatively, camera 30 may also directly transmit data via any forms of wireless communication to a processor as required by the procedure performed by endoscope 100.

Referring to FIGS. 1, 8 and 9, handle 48 may contain a small electronics circuit board (not shown) which includes a processor on a circuit board (not shown) that processes the image data capture by an image sensor or camera 30 at the tip of the endoscope. The processor coverts the image data from the format output to the standard image format such as HDMI that can be displayed on typical monitors.

Camera 30 at the end of distal tip may be connected with the circuit board via electrical wire 32 which, together with optical fiber and working channel (which may pass fluids directly or with a conduit, working tool, etc.), goes through the flexible probe as well as vertebra column 12 to reach circuit board. Camera 30 may digitize optical data and transmits digitized data to circuit board 48 for processing. Further alternatively, camera 30 may be in direct wireless communication with a camera control unit (CCU) (not shown). All such variations are within the scope of the present disclosure.

Referring to FIGS. 8-9, in one embodiment, the end surface of distal tip 16, perpendicular to the axial direction, may include one or more light sources or an opening of the fiber cable 28 conducting light from the light sources, disposed in a similar fashion as camera 30. Working channel 20 also has an opening (not shown) at the end of tip 16. The relative placement of any of these components can be varied to suit a particular application. The light source, if placed in handle 48, is connected via an optical fiber housed within optical fiber cable 28, going through vertebra column 12 and reaches distal tip 16.

Working channel may be pre-installed in the probe, through probe conduit, continuing into vertebra column 12, with working channel 20 molded at the same time when vertebra column 12 is molded or made with plastic extrusion. In some embodiments, other kind of optical fibers may be introduced to the working channel 20 during a procedure to, for example, introduce laser energy for blasting a target area. Suitable tool(s) can also extend through the working channel 32a or through specific conduits other than the working channel to, for example, assist the fragmentation of kidney stones. Surgical tools such as collapsible basket for capturing kidney stones can also be threaded though working channel 32a.

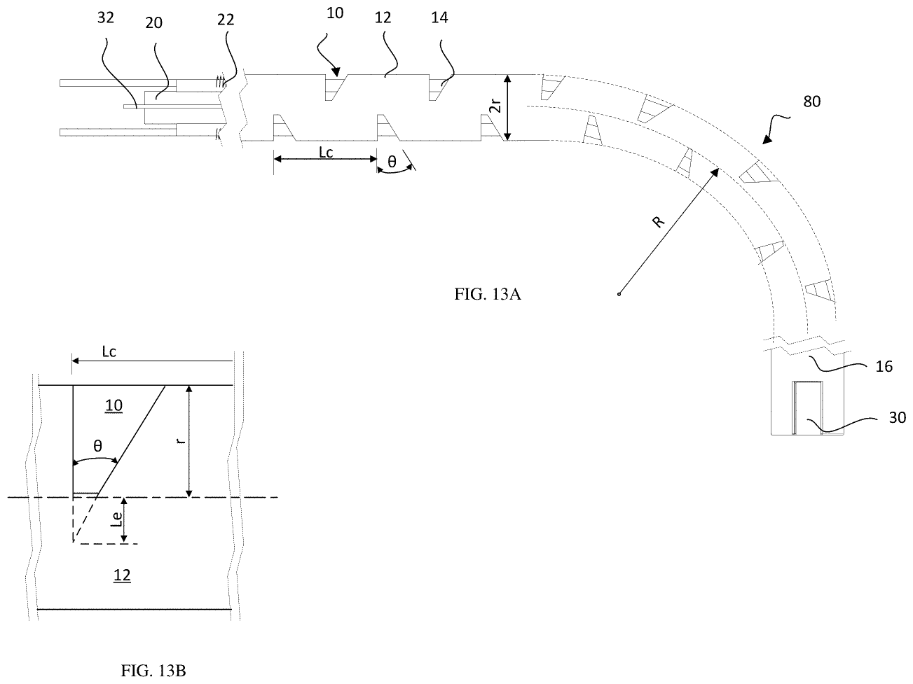

FIG. 13A is a side view of the elongated vertebra column of the endoscope with a bent distal tip in accordance with of the present disclosure.

As can be seen in FIG. 13, when vertebrae column 12 is bent or deflected to a shape of an arch 80.

FIG. 13B is an elaborated view of the vertebrae column 12 surrounding one vertebrae slit 10.

Referring to FIGS. 13 A and 13B, in one example embodiment, assuming the diameter of the vertebrae column 12 is 2r, a cutting angle is θ, and the distance between the two adjacent vertebrae slits is Lc, and a depth of the slit is (Le+r), one can have a “maximum bent” or minimum radius of arch R calculated as:

$$R = \frac{L c - ( r + Le ) \cdot tan ( θ )}{θ} \qquad Eq . 1$$

One can see that the depth of the slit 10, (r+Le), includes a depth of imaginary lines extended from the physical cut of vertebrae slit 10.

Assuming a length of a flex section of the vertebrae column 12, is S, the total number of vertebrae slits in one group of the vertebrae slits 10, n, is given by:

$$n = \frac{S}{L c} \qquad Eq . 2$$

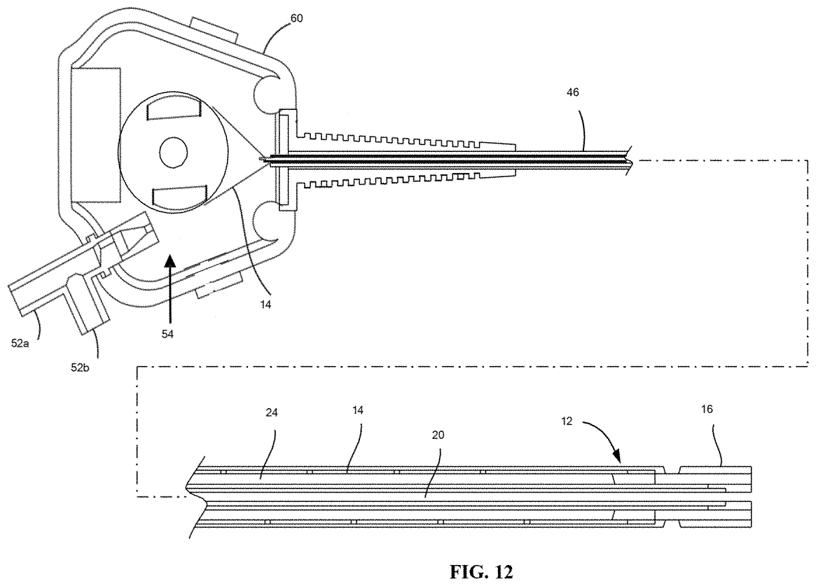

Still referring to FIGS. 11 and 13A, a flexion angle φ can be expressed as:

$$φ = \frac{S}{R} \qquad Eq . 3$$

Those skilled in the art should appreciate the advantage of one of the novel aspects to have at least two parallel groups of gaping slits aligned longitudinally along the vertebrae column, on the opposite side of each other. Each two adjacent vertebrae slits being arranged in a staggered fashion along the longitudinal (axial) direction of the vertebrae column allows a deeper cut of the gaping slits 10 without compromise the strength and the stiffness the vertebrae column. Subsequently it serves the purpose of providing easier bending and more agility of the surgical operation.

One can also see in FIG. 13A that vertebrae slits 10 on the outside of arch 80 widen while slits 10 on the inside of arch 80 become narrower to a degree to accommodate the deflection. This configuration also serves the purpose of providing easier bending and more agility of the endoscopy operation.

In some embodiment, a circumferential length of the gaping slits is preferably less than or equal to half of a circumference of the vertebra column, and larger than 1/16 of the circumference of the vertebra column.

In some embodiment, a depth of the gaping slits is less than half of a diameter of the vertebra column, and larger than 1/16 of the diameter of the vertebra column.

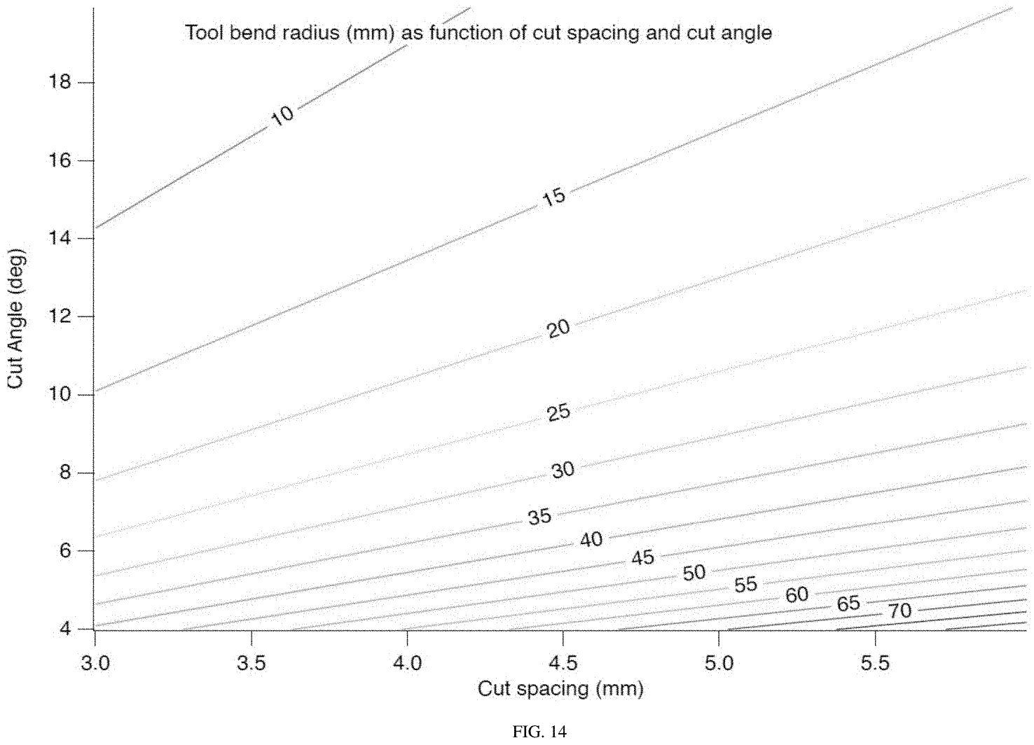

FIG. 14 is a diagram showing the sizing of the vertebrae slit and the bending of the distal tip of the endoscope in accordance with of the present disclosure. It shows an example relationship among the diameter of achieved bent radius R, cutting angle θ, and the distance between the two adjacent vertebrae is Lc (cut spacing). It can be seen that the larger the cut angles, the more degree of the deflection (smaller the radius R) one can achieve for the vertebrae column 12.

Additionally, it is contemplated that systems, devices, methods, and processes of the present application encompass variations and adaptations developed using information from the embodiments described in the following description. Adaptation or modification of the methods and processes described in this specification may be performed by those of ordinary skill in the relevant art.

Throughout the description, where compositions, compounds, or products are described as having, including, or comprising specific components, or where processes and methods are described as having, including, or comprising specific steps, it is contemplated that, additionally, there are articles, devices, and systems of the present application that consist essentially of, or consist of, the recited components, and that there are processes and methods according to the present application that consist essentially of, or consist of, the recited processing steps.

It should be understood that the order of steps or order for performing certain action is immaterial so long as the described method remains operable. Moreover, two or more steps or actions may be conducted simultaneously.

What is claimed:

1. An endoscope comprising:
an elongated probe having a distal end, a proximal end, and a vertebrae column immediately abutting the distal end;
a handle abutting the proximal end of the probe and comprising an articulation lever;
wherein the vertebrae column is configured to have at least two parallel groups of gaping slits, along an axial direction of the vertebrae column, wherein each two axially adjacent gaping slits come from the two groups of gaping slits respectively, are juxtaposed in circumferential positions in the respective circumferential planes; and
wherein the articulation lever and the distal end are connected by pulling wires, and in response to the articulation lever being maneuvered, the distal end is pulled away from the axial direction, causing the vertebrae column to deflect.

2. The endoscope of claim 1, wherein the vertebra column probe is an elongated hollow body providing passageway for the at least one of an optical transmission, a pull wire channel, an electrical wire, and a working channel.

3. The endoscope of claim 2, wherein the vertebra column and at least the working channel and the pull wire channel are made by the same process of plastic extrusion.

4. The endoscope of claim 2, wherein the vertebra column and at least the working channel and the pull wire channel are made by the same process of plastic molding.

5. The endoscope of claim 1, wherein a circumferential length of the gaping slits is less than or equal to half of a circumference of the vertebra column, and larger than 1/16 of the circumference of the vertebra column.

6. The endoscope of claim 1, wherein a depth of the gaping slits is less than half of a diameter of the vertebra column, and larger than 1/16 of the diameter of the vertebra column.

7. The endoscope of claim 1, wherein the vertebrae column is defected to form an arch with a radius of R, wherein R=(Lc−(r+Le)·tan(θ))/θ, (r+Le) is related to a depth of the gaping slits, Lc is a distance between the two adjacent gaping slits of the same group, and θ is the cut angle of the gaping slits.

8. The endoscope of claim 7, wherein a flexion angle φ is determined by
$$φ = \frac{S}{R} ,$$
wherein S is a total length go the vertebrae column.

9. The endoscope of claim 1, wherein a total number of the gaping slits in one group is determined by
$$n = \frac{S}{L c} ,$$
wherein S is a total length of the vertebrae column and Lc is a distance between the two adjacent gaping slits of the same group.

10. The endoscope of claim 1, wherein the gaping slits are machined onto a circumferential surface of the vertebra column.

11. The endoscope of claim 1, wherein the vertebra column is a round cylinder.

12. The endoscope of claim 1, wherein the vertebra column is a straight prism.

13. An elongated vertebrae column configured to have at least two parallel groups of gaping slits along an axial direction of the vertebrae column with each two axially adjacent gaping slits coming from one of the two groups of gaping slits respectively, the two adjacent gaping slits are juxtaposed in circumferentially positions in respective circumferential planes,
wherein the vertebrae column is part of an elongated probe of an endoscope, the probe has a sensor at a distal end abutting one end of the vertebra column, and the probe is attached to a handle at a proximal end of the probe, the handle has an the articulation lever which is connected with the distal end by at least one pulling wire, and when the articulation lever is maneuvered, the distal end is pulled away from the axial direction, causing the vertebrae column to deflect.

14. The endoscope of claim 13, wherein the vertebra column probe is an elongated hollow body providing passageway for the at least one of an optical transmission, a pull wire channel, an electrical wire, and a working channel.

15. The endoscope of claim 14, wherein the vertebra column and at least the working channel and the pull wire channel are made by the same process of plastic protrusion.

16. The endoscope of claim 14, wherein the vertebra column and at least the working channel and the pull wire channel are made by the same process of plastic molding.

17. The endoscope of claim 13, wherein the vertebrae column is defected to form an arch with a radius of R, S is a total length go the vertebrae column, a flexion angle φ is determined by
$$φ = \frac{S}{R} .$$

18. The endoscope of claim 13, wherein the gaping slits are machined onto a circumferential surface of the vertebra column.

19. The endoscope of claim 13, wherein the vertebra column is a round cylinder.

20. The endoscope of claim 13, wherein the probe distal end hosts an optical sensor, an opening for the working channel, and at least one light source.

21. The endoscope of claim 13, wherein the sensor is a camera providing data to be transmitted by the electrical transmission.
